(19) United States
(12) Patent Application Publication
(10) Pub. No.: US 2025/0325470 A1
(43) Pub. Date: Oct. 23, 2025

(54) METHOD FOR PRODUCING FERMENTED CAVIAR OIL USING SKIN FLORA, AND COSMETIC COMPOSITION COMPRISING SAME

(71) Applicants: COSMAX, INC., Gyeonggi-do (KR); ALMASCAVIAR CO., LTD., Gyeonggi-do (KR)

(72) Inventors: Kwang Nyeon KIM, Seoul (KR); Hyun Tak HAN, Seoul (KR); Ju Hyun SON, Seoul (KR); Sa Rang CHO, Seoul (KR); Ju Yun KIM, Seoul (KR); Tae Hyun KIM, Seoul (KR); Sugyeong JEONG, Seoul (KR); Seok Kyun YUN, Gyeonggi-do (KR); Yae Rin LEE, Gyeonggi-do (KR); Seung Hyun KANG, Seoul (KR); Myeong Sam PARK, Seoul (KR); Youn Hwa NHO, Seoul (KR); Sang Hun HAN, Gyeonggi-do (KR); Jehee HAN, Gyeonggi-do (KR)

(21) Appl. No.: 18/707,874

(22) PCT Filed: Oct. 7, 2022
(86) PCT No.: PCT/KR2022/015104

§ 371 Date: Oct. 9, 2024

(30) Foreign Application Priority Data
Nov. 9, 2021 (KR) 10-2021-0152921

(51) Int. Cl. A61K 8/92; A61Q 19/00; C12N 1/20; C12R 1/01

(52) U.S. Cl. CPC A61K 8/925; A61Q 19/00; C12N 1/205; A61K 2800/85; C12R 2001/01

(57) ABSTRACT

The present invention relates to a fermented oil in which the content of a lipid fermentation metabolite having an amine group is increased by fermenting caviar and vegetable oil with skin flora, and a cosmetic composition comprising same. The cosmetic composition according to the present invention can improve anti-aging, barrier strengthening, moisturizing, and differentiation effects, thereby maximizing beneficial effects on the skin.

### TECHNICAL FIELD

The present invention relates to a method for producing fermented oil in which the content of a lipid fermentation metabolite having an amine group is increased by fermenting caviar and vegetable oil with skin flora, and a cosmetic composition comprising the fermented oil.

### BACKGROUND ART

The skin is composed of the epidermis, dermis, and subcutaneous fat layers. Among them, the epidermis exists in the outermost layer, serves as a protective film for the skin, and is responsible for the immune function of the skin. The skin is inhabited by skin flora, and these microorganisms are mainly found in the outermost layer of the epidermis and the upper part (upper layer) of hair follicles. There are about 1,000 kinds of microorganisms as the skin flora, and among them, aerobic microorganisms can secrete lipolytic enzymes that can utilize lipids in the epidermal layer.

The epidermal layer of the skin is mainly composed of keratinocytes, lipid components are formed between the keratinocytes, and the outermost layer is composed of protein. The stratum corneum of the epidermis contains about 10 to 20% of moisture and exists in the outermost layer of the human body, thereby suppressing the evaporation of moisture to the outside of the body while blocking the excessive penetration of substances from the outside. These stratum corneum surfaces can be surrounded by a thin natural protective film made of sebum from sebaceous glands and sweat from sweat glands, thereby preventing the evaporation of moisture. The cells that make up this stratum corneum contain a high concentration of natural moisturizing factor (NMF), a water-soluble component, which helps maintain skin flexibility and appropriate moisture. For example, substances such as amino acids are not only water-soluble, but also effectively bind with moisture, thereby preventing the skin moisture from drying out.

Meanwhile, sturgeon eggs are rich in nutrients such as amino acids and fats, and are known to show the effects of regenerating epidermal lipid membranes and preventing aging, so they are sometimes included as a raw material in cosmetics.

As moisture in the stratum corneum decreases due to various causes such as aging, and stress caused by various stimuli such as artificial temperature control of cooling/heating, changes in the external environment such as environmental pollution, or changes in lifestyle patterns, the skin becomes dry, the skin surface becomes rough, and the skin loses its luster, which has a negative effect on the skin in terms of beauty and skin health, thereby increasing the need for skin moisturizers.

The present invention seeks to provide a fermented oil production method capable of generating a lipid fermentation metabolite having an amine group and improving skin health by using caviar as a nitrogen source when fermenting skin flora and vegetable oil, and a composition including the fermented oil.

### DISCLOSURE OF THE INVENTION

### Technical Goals

An object of the present invention is to provide a cosmetic composition in which the content of a lipid fermentation metabolite having an amine group is increased by fermenting caviar and vegetable oil with skin flora, and a method for producing the same.

### Technical Solutions

In order to solve the above problems, the present invention provides a method for producing a cosmetic composition containing fermented caviar oil, the method including:
a seed culture step of producing a seed culture by culturing microorganisms with deposit number of KCCM 11843P (Korean Culture Center of Microorganisms (Overseas), 2016.06.08) as skin flora;
a preculture step of culturing the seed culture to produce a preculture; and
a main fermentation step of producing a fermented product by adding the preculture to a medium containing caviar extract.

According to one embodiment, the medium of the seed culture step or the medium of the preculture step may contain casein hydrolyzate, yeast extract, glucose, soluble starch, K₂HPO₄, MgSO₄, sodium pyruvate, and casein peptone.

Specifically, for example, the medium of the seed culture step or the medium of the preculture step may contain 0.1 to 5 g/L of casein hydrolysate, 0.01 to 10 g/L of yeast extract, 0.1 to 10 g/L of glucose, 0.1 to 5 g/L of soluble starch, 0.1 to 5 g/L of K₂HPO₄, 0.1 to 10 g/L of MgSO₄, 0.05 to 5 g/L of sodium pyruvate, and 0.1 to 5 g/L of casein peptone.

According to one embodiment, the medium of the main fermentation step may contain glycerol, yeast extract, casein peptone, K₂HPO₄, KH₂PO₄, and ammonium acetate.

Specifically, for example, the medium of the main fermentation step may contain 1 to 100 g/L of glycerol, 0.01 to 10 g/L of yeast extract, 0.1 to 10 g/L of casein peptone, 0.5 to 50 g/L of K₂HPO₄, 0.3 to 30 g/L of KH₂PO₄, 0.005 to 0.1 g/L of ammonium acetate, and 5 to 250 g/L of caviar extract.

According to one embodiment, the medium of the main fermentation step may further contain vegetable oil.

Specifically, for example, the content of the vegetable oil may be 5 to 500 g/L.

According to one embodiment, the seed culture step may include shaking culturing under aerobic conditions of 15 to 35° C.,

The preculture step may include adding 10 to 500 g/L of the seed culture and culturing under aerobic conditions of 15 to 35° C., 5 to 20 NL/min, and 10 to 100 rpm, and

The culture in the preculture step may be terminated when the spectrophotometric absorbance of 5% culture solution at a wavelength of 600 nm is 0.2 to 0.8.

According to one embodiment, the main fermentation step may include adding to 500 g/L of the preculture and fermenting it for 50 to 150 hours under aerobic conditions of 15 to 35° C., 100 to 500 NL/min, and 100 to 300 rpm.

According to one embodiment, the composition may be a composition for skin anti-aging, skin barrier strengthening, or skin moisturizing.

According to another embodiment of the present invention, there is provided a cosmetic composition containing fermented caviar oil prepared by the method described above.

Specific matters of other embodiments according to the present invention are presented in the detailed description below.

### Advantageous Effects

The cosmetic composition according to the present invention has enhanced activity and content of active substances through fermentation of caviar and vegetable oil using skin flora, thereby improving anti-aging, barrier strengthening, moisturizing and differentiation effects to maximize beneficial effects on the skin.

### BRIEF DESCRIPTION OF DRAWINGS

FIG. 1 is a photograph of a fermented product observed with the naked eye.

FIG. 2 is a graph analyzing the expression levels of MMP1.

FIG. 3 is a graph analyzing the expression levels of COL1A1.

FIG. 4 is a graph analyzing the expression levels of HAS3.

FIG. 5 is a graph analyzing the expression levels of Aquaporin3.

FIG. 6 is a graph analyzing the expression levels of Claudin.

FIG. 7 is a graph analyzing the expression levels of Filaggrin.

FIG. 8 is a graph analyzing the expression levels of Involucrin.

FIG. 9 is a graph analyzing the expression levels of Loricrin.

### BEST MODE FOR CARRYING OUT THE INVENTION

Although the present invention may be modified in various ways and have various embodiments, specific embodiments will be exemplified and described in detail. However, the present invention is not intended to be limited to the specific embodiments, and should be understood to include all modifications, equivalents, and substitutes included in the spirit and technical scope of the present invention. In describing the present invention, if it is determined that detailed descriptions of related known technologies may obscure the gist of the present invention, the detailed description thereof will be omitted.

Hereinafter, a method for producing fermented caviar oil according to the present invention and a cosmetic composition containing the fermented caviar oil will be described in detail.

The present invention provides a composition with improved skin efficacy and effect due to an increase in fermentation metabolites that produce fermented oil through the fermentation of caviar extract and vegetable oil using skin flora which has a beneficial effect on the skin and coexists among the various microorganisms that inhabit the skin.

Specifically, the present invention provides a method for producing a cosmetic composition containing fermented caviar oil, the method including:
a seed culture step of producing a seed culture by culturing microorganisms with deposit number of KCCM 11843P (Korean Culture Center of Microorganisms (Overseas), 2016.06.08) as skin flora;
a preculture step of culturing the seed culture to produce a preculture; and
a main fermentation step of producing a fermented product by adding the preculture to a medium containing caviar extract.

The skin flora of the present invention is a strain of Epidermidibacterium keratini sp.

Caviar is salted roe of sturgeon and is well known as a luxurious food ingredient called one of the world's top three delicacies. It is rich in various nutrients such as protein, fat, sugar, and vitamins, and essential amino acids such as leucine and arginine, as well as various amino acids such as glutamic acid, serine, and alanine. In addition, the fat contained in caviar is mostly composed of cholesterol and lecithin, which can help regenerate the lipid membrane of the epidermis, and is rich in omega-3 fatty acids, thereby providing an excellent anti-aging effect. The caviar extract of the present invention may include caviar hydrolyzate. The caviar hydrolyzate can be produced using a protease.

Fermented caviar oil can be obtained by fermenting caviar and vegetable oil as a substrate with skin flora. The fermented caviar oil contains skin-like lipids with amine groups, fermented emulsifiers, unsaturated fatty acids, and various other metabolites.

According to one embodiment, the medium of the seed culture step or the medium of the preculture step may contain casein hydrolyzate, yeast extract, glucose, soluble starch, K₂HPO₄, MgSO₄, sodium pyruvate, and casein peptone.

Specifically, the medium of the seed culture step or the medium of the preculture step may contain 0.1 to 5 g/L, such as 0.1 to 3 g/L, such as 0.1 to 1 g/L, of casein hydrolysate, 0.01 to 10 g/L, such as 0.1 to 5 g/L, such as 0.1 to 3 g/L, such as 0.1 to 1 g/L, of yeast extract, 0.1 to 10 g/L, such as 0.1 to 5 g/L, such as 0.1 to 3 g/L, such as 0.1 to 1 g/L, of glucose, 0.1 to 5 g/L, such as 0.1 to 3 g/L, such as 0.1 to 1 g/L, of soluble starch, 0.1 to 5 g/L, such as 0.1 to 3 g/L, such as 0.1 to 1 g/L, such as 0.1 to 0.5 g/L, of K₂HPO₄, 0.1 to 10 g/L, such as 0.1 to 5 g/L, such as 0.1 to 1 g/L, of MgSO₄, 0.05 to 5 g/L, such as 0.1 to 3 g/L, such as 0.1 to 1 g/L, such as 0.1 to 0.5 g/L, of sodium pyruvate, and 0.1 to 5 g/L, such as 0.1 to 3 g/L, such as 0.1 to 1 g/L, of casein peptone.

According to one embodiment, the medium of the main fermentation step may contain glycerol, yeast extract, casein peptone, K₂HPO₄, KH₂PO₄, ammonium acetate, and caviar extract.

Specifically, the medium of the main fermentation step may contain 1 to 100 g/L, such as 1 to 50 g/L, such as 1 to 30 g/L, such as 1 to 20 g/L, of glycerol, 0.01 to 10 g/L, such as 0.01 to 0.5 g/L, such as 0.05 to 0.3 g/L, of yeast extract, 0.1 to 10 g/L, such as 0.1 to 5 g/L, such as 0.5 to 3 g/L, of casein peptone, 0.5 to 50 g/L, such as 0.5 to 30 g/L, such as 1 to 10 g/L, of K₂HPO₄, 0.3 to 30 g/L, such as 0.3 to 20 g/L, such as 1 to 10 g/L, such as 1 to 5 g/L, of KH₂PO₄, 0.005 to 0.1 g/L, such as 0.001 to 0.05 g/L, such as 0.01 to 0.1 g/L, of ammonium acetate, and 5 to 250 g/L, such as 50 to 250 g/L, such as 50 to 200 g/L, of caviar extract.

According to one embodiment, the medium of the main fermentation step may further contain vegetable oil. Specifically, the content of vegetable oil may be 5 to 500 g/L, such as 100 to 500 g/L, such as 200 to 300 g/L, such as 300 to 500 g/L.

According to one embodiment, the vegetable oil may include one or more of edible or human-friendly oils such as macadamia oil, sunflower seed, grape seed, canola, rice germ, olive, soybean, argan, brown rice, perilla seed, sesame seed, almond, peanut, corn, red ginseng, avocado, macadamia, coconut, rose hip, vitamin tree seed, shea tree fruit, oil palm, bergamot fruit, camellia seed, safflower seed, apricot seed, poppy seed, evening primrose seed, castor seed, green tea seed, meadowfoam seed, flax seed and hemp seed, but is not particularly limited thereto.

According to one embodiment, the seed culture step may include shaking culturing under aerobic conditions of 15 to 35° C., such as 15 to 30° C.

In addition, the preculture step may include adding 10 to 500 g/L, such as 50 to 300 g/L, 50 to 200 g/L, 50 to 150 g/L, of the seed culture and culturing under aerobic conditions of 15 to 35° C., such as 15 to 25° C., 5 to 20 NL/min, such as 10 to 20 NL/min, and 10 to 100 rpm, such as 30 to 80 rpm.

In addition, the culture in the preculture step may be terminated when the spectrophotometric absorbance of the culture solution diluted to 5% at a wavelength of 600 nm is 0.2 to 0.8, such as 0.2 to 0.4.

According to one embodiment, the main fermentation step may include adding to 500 g/L, such as 50 to 300 g/L, 50 to 200 g/L, 50 to 150 g/L, of the preculture and fermenting it for 50 to 150 hours, such as 50 to 100 hours, 60 to 80 hours under aerobic conditions of 15 to 35° C., such as 15 to 25° C., 100 to 500 NL/min, such as 100 to 300 NL/min, 200 to 400 NL/min, and 100 to 300 rpm, such as 100 to 200 rpm.

According to one embodiment, separation and purification steps may be further included after the main fermentation step. For example, a step of centrifuging the main fermented product to separate the oil layer and the water layer, and then recovering the oil layer may be included. In addition, filtration using a filter can be applied by adding MgSO₄ to remove residual moisture and impurities, but the method applied for purification is not limited to the above.

The present invention can maximize the effect of the active substances contained in caviar with the above-described configuration. Since the active substances included in the present invention can provide beneficial help to skin cells, they are suitable for application to compositions for skin anti-aging, skin barrier strengthening, or skin moisturizing. Specifically, the present invention can maintain healthy skin by helping to regenerate damaged skin cells, such as keratinocyte, melanocyte, and fibroblasts present in the dermis and responsible for the biosynthesis of collagen and elastin.

According to one embodiment, the cosmetic composition of the present invention may include components commonly used in cosmetic compositions, such as stabilizers, solubilizers, vitamins, pigments, flavors, adjuvants, and carriers.

In addition, it may be prepared in any formulation that is commonly manufactured in the art and is dermatologically applicable.

‘Dermatologically applicable’ may mean that a composition can have an effective action that is relatively non-toxic and harmless to the subject to which it is applied, and may include an external agent applicable to the skin, wherein side effects resulting from the composition do not reduce the efficacy of the active ingredients, do not cause serious irritation to the applied subject, and do not impair the activity and properties of the active ingredients. The dermatologically applicable cosmetic composition of the present invention may be formulated, for example, as a solution, suspension, emulsion, emulsified liquid, paste, gel, pack, cream, lotion, powder, soap, surfactant-containing cleansing, oil, powder foundation, emulsion foundation, wax foundation, spray, and hair cosmetic, but is not limited thereto.

Specifically, it may be manufactured as a formulation of skin lotion, skin softener, skin toner, astringent, lotion, gel, milk lotion, moisture lotion, nutritional lotion, massage cream, nutritional cream, moisture cream, hand cream, foundation, essence, ampoule, nutritional essence, pack, soap, hair shampoo, foot shampoo, cleansing foam, cleansing lotion, cleansing cream, body lotion, and body cleanser.

Hereinafter, examples of the present invention will be described in detail so that those skilled in the art can easily implement the present invention. However, the present invention may be embodied in a variety of forms and is not limited to the examples described herein.

### Preparation Example 1: Preparation of Caviar Extract

To prepare a caviar hydrolyzate, caviar (purchased from Almas Caviar) was ground with a blender. The ground caviar and 0.1M sodium phosphate (pH 7.0) were mixed at a ratio of 1:1 (w/v). After raising the temperature of the mixture to 35° C., 0.05% of bromelain, a proteolytic enzyme, was added and reacted for 2 hours while stirring. After the reaction, a caviar extract was prepared by losing enzyme activity at 90° C. for 15 minutes.

### Example 1: Production of Fermented Caviar Oil

### Seed Culture

As skin flora, a microorganism with deposit number of KCCM 11843P (Korean Culture Center of Microorganisms (Overseas), 2016.06.08) was seed cultured. It was inoculated into R2A (BD Difco, USA) medium, and shaking culture was performed under aerobic conditions at 25° C. to prepare a seed culture. Specifically, the medium has a composition of 0.5 g/L of casein acid hydrolysate, 0.5 g/L of yeast extract, 0.5 g/L of glucose, 0.5 g/L of soluble starch, 0.3 g/L of K₂HPO₄, 0.5 g/L of MgSO₄, 0.3 g/L of sodium pyruvate, and 0.5 g/L of casein peptone.

### Preculture

100 g/L of the seed culture was added to a medium having the same composition as in the seed culture step, and cultured under aerobic conditions at 20° C., 15 NL/min, and 50 rpm to prepare a preculture.

For the preculture, the inoculation point of the main fermentation was set by turbidity measurement. Specifically, the preculture was sampled and diluted to 5% with 0.85% NaCl solution, and then the absorbance was measured at a wavelength of 600 nm using a spectrophotometer (BioTek, USA). The end point of preculture and the inoculation point of main fermentation were set based on the absorbance range of 0.2 to 0.4.

### Main Fermentation

100 g/L of the preculture was added to a medium having the composition shown in Table 1 when the temperature was 20° C., and fermented for 72 hours under aerobic conditions at 20° C., 300 NL/min, and 150 rpm to produce a fermented product (fermented oil). The caviar extract in Table 1 was prepared from Preparation Example 1.

TABLE 1

| | Comparative | | | |
|---|---|---|---|---|
| Classification (g/L) | Example 1 | Example 1 | Example 2 | Example 3 |
| Glycerol | 10.0 | 10.0 | 10.0 | 10.0 |
| Yeast extract | 0.1 | 0.1 | 0.1 | 0.1 |
| Casein peptone | 1.0 | 1.0 | 1.0 | 1.0 |
| K₂HPO₄ | 4.5 | 4.5 | 4.5 | 4.5 |
| KH₂PO₄ | 3.0 | 3.0 | 3.0 | 3.0 |
| Ammonium acetate | 0.02 | 0.02 | 0.02 | 0.02 |
| Caviar extract | — | 50.0 | 100.0 | 200.0 |
| Macadamia oil | 500.0 | 500.0 | 500.0 | 500.0 |
| Purified water | 481.38 | 431.38 | 381.38 | 281.38 |

### Separation and Purification

In order to obtain fermented oil from the fermented product, the fermented product was centrifuged to separate an oil layer. The oil layer was recovered, and 100 g/L of MgSO₄ was added and then stirred to remove remaining moisture and impurities. After stirring for 2 hours, it was filtered using a filter pad (CH-ST-150, Hyundai Micro, Korea) in a filter press. The filtered oil was finally sterilized and filtered (0.2 μm filter) to obtain a fermented caviar oil.

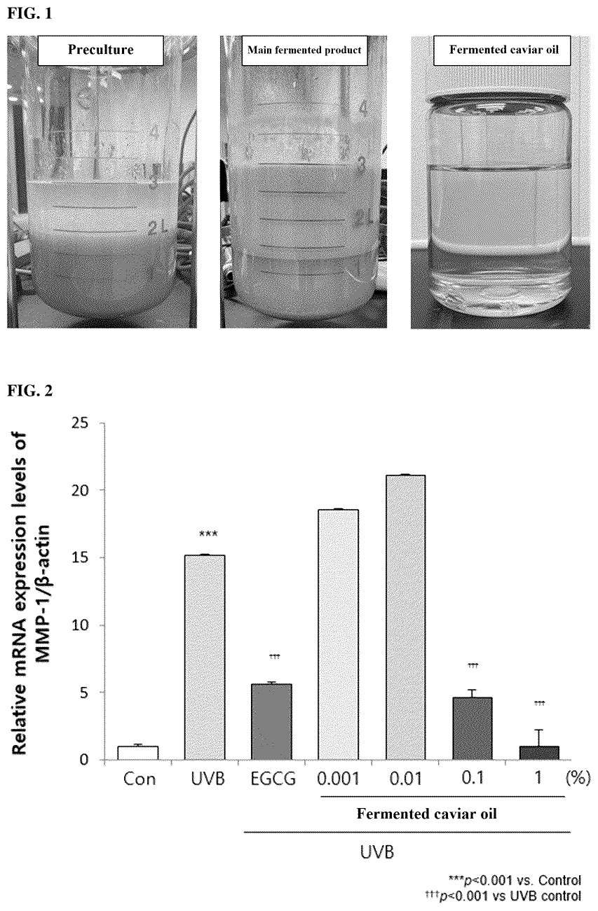

Visual observation photographs of the preculture before the main fermentation, the main fermented product before purification, and the fermented caviar oil as the final obtained product are shown in FIG. 1.

### Experimental Example 1: Evaluation of MMP-1 Inhibition Ability

To compare the inhibitory ability of fermented caviar oil against matrix metalloproteinase (MMP-1), which is increased by ultraviolet rays, MMP-1 expression level was measured using human fibroblast cell line Hs68.

The matrix metalloproteinases (MMPs) are representative enzymes that decompose matrix proteins. The dermis plays a role in supporting the strength and shape of the skin, and most of the matrix proteins are present in the dermis. When the matrix proteins are decomposed by MMPs, wrinkles, decreased skin elasticity, skin sagging, and dryness occur. MMPs act as a major factor in accelerating skin aging because their activity increases even with a single UV irradiation.

In order to evaluate the MMP-1 inhibition ability, the fibroblast cell line Hs68 was dispensed into a 6-well plate at an amount of 3×10⁵ cell/well and then cultured in an incubator at 37° C. and 5% CO₂ for 24 hours. Thereafter, the medium was removed, Dulbecco's phosphate-buffered saline (DPBS) was added, the remaining cell groups, excluding the non-UVB irradiated group, were irradiated with 20 mJ/cm² of UVB, and then the fermented caviar oil according to Example 1 was added and further cultured for 24 hours. In this case, 1 μM of EGCG was used as a positive control. Thereafter, RNA was isolated from cells treated with each sample by using Trizol (RNA iso, DAKARA, Japan), RNA was quantified by using nanodrop, and then cDNA was synthesized in an amplifier using 2 μg of RNA, respectively (C1000 Thermal Cycler, Bio-Rad, USA). Real-time polymerase chain reaction was performed on a real-time PCR machine using a mixture of an MMP-1 primer, a target protein, and SYBR Green (SYBR Green supermis, Applied Biosystems, USA), a cyanine dye, added to the synthesized cDNA, thereby finally evaluating the expression level of the MMP-1 gene.

The sequences and reaction conditions of the primer are shown in Table 2, and the gene expression levels were finally analyzed through correction for the β-actin gene.

TABLE 2

| Primer | | Sequence | Reaction condition |
|---|---|---|---|
| MMP-1 | F | 5′-CGAATTGCCGACAGAGATGA-3′ | After polymerase activation at |
| | R | 5′-GTCCCTGAACAGCCCAGTACTT-3′ | 94° C. for 5 minutes, |
| β-actin | F | 5′-GGCCATCTCTTGCTCGAAGT-3′ | polymerization reaction was |
| | R | 5′-GAGACCTTCAACACCCCAGC-3′ | performed in 40 cycles under |
| | | | conditions of 95º C. for 30 |
| | | | seconds, 55° C. for 30 seconds, and |
| | | | 72º C. for 30 seconds. |

As a result, as shown in FIG. 2, it was confirmed that the expression level of MMP-1 increased rapidly by UVB irradiation, whereas the expression level decreased in the fermented caviar oil-treated group.

### Experimental Example 2: Confirmation of Procollagen Expression

In order to confirm the anti-wrinkle effect of the fermented caviar oil according to Example 1, the fibroblast cell line Hs68 was dispensed into a 6-well plate at an amount of 3×10⁵ cell/well and then cultured in an incubator at 37° C. and 5% CO₂ for 24 hours. Thereafter, the medium was removed, DPBS was added, the remaining cell groups, excluding the non-UVB irradiated group, were irradiated with 20 mJ/cm² of UVB, and then the fermented caviar oil was added and further cultured for 24 hours. In this case, 1 μM of EGCG was used as a positive control. Thereafter, RNA was isolated from cells treated with each sample by using Trizol (RNA iso, DAKARA, Japan), RNA was quantified by using nanodrop, and then cDNA was synthesized in an amplifier using 2 μg of RNA, respectively (C1000 Thermal Cycler, Bio-Rad, USA). Real-time polymerase chain reaction was performed on a real-time PCR machine using a mixture of a procollagen primer, a target protein, and SYBR Green (SYBR Green supermis, Applied Biosystems, USA), a cyanine dye, added to the synthesized cDNA, thereby finally evaluating the expression level of the type 1 procollagen (Col1A1) gene. The sequences and reaction conditions of the primer are shown in Table 3, and the gene expression levels were finally analyzed through correction for the β-actin gene.

TABLE 3

| Primer | | Sequence | Reaction condition |
|---|---|---|---|
| type 1 | F | 5′-CTCGAGGTGGACACCACCCT-3′ | After polymerase activation at |
| procollagen | R | 5′-CAGCTGGATGGCCACATCGG-3′ | 94° C. for 5 minutes, |
| β-actin | F | 5′-GGCCATCTCTTGCTCGAAGT-3′ | polymerization reaction was |
| | R | 5′-GAGACCTTCAACACCCCAGC-3′ | performed in 40 cycles under |
| | | | conditions of 95° C. for 30, |
| | | | seconds 55º C. for 30 seconds, and |
| | | | 72° C. for 30 seconds. |

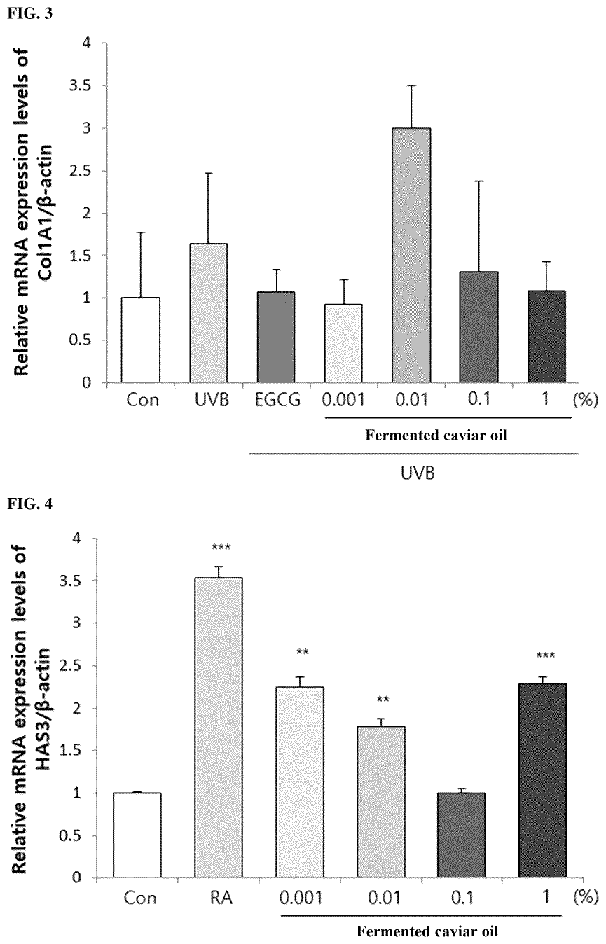

As a result, as shown in FIG. 3, it was confirmed that the production of type 1 procollagen (Col1A1) increased when skin cells were treated with the fermented caviar oil, and through this, the effect of improving skin elasticity and wrinkles was confirmed.

### Experimental Example 3: Confirmation of Moisturizing Effect

The moisturizing effect of the fermented caviar oil according to Example 1 was confirmed. The expression level of hyaluronic acid synthase occurring in HaCaT cells was confirmed by hyaluronic acid synthase 3 (HAS3), and the expression level of intracellular water channels was evaluated by measuring aquaporin 3 (AQP3) gene expression.

The aquaporins are a transmembrane protein (major intrinsic protein family) that selectively passes only water molecules and exists in all cell membranes, and have a mechanism to increase moisture absorption in the skin. There are 13 types known in mammals, and among them, aquaporin 3 is expressed in keratinocytes of the skin, and the absorption and movement of water and glycerol through the aquaporin 3 prevents moisture loss from cells, thereby increasing the moisturizing and elasticity of the skin.

In order to confirm the moisturizing effect, keratinocyte HaCaT was dispensed into a 6-well plate at an amount of 4×10⁵ cell/well and then cultured in an incubator at 37° C. and 5% CO₂ for 24 hours. Thereafter, the fermented caviar oil was added and further cultured for 24 hours. In this case, 1 μM of retinoic acid (RA) was used as a positive control. Thereafter, RNA was isolated from cells treated with each sample by using Trizol (RNA iso, DAKARA, Japan), RNA was quantified by using nanodrop, and then cDNA was synthesized in an amplifier using 2 μg of RNA, respectively (C1000 Thermal Cycler, Bio-Rad, USA).

Real-time polymerase chain reaction was performed on a real-time PCR machine using a mixture of a HAS3 or AQP3 primer, a target protein, and SYBR Green (SYBR Green supermis, Applied Biosystems, USA), a cyanine dye, added to the synthesized cDNA, thereby finally evaluating the expression level of the HAS gene and AQP3 gene. The sequences and reaction conditions of the primer are shown in Table 4, and the gene expression levels were finally analyzed through correction for the β-actin gene.

TABLE 4

| Primer | | Sequence | Reaction condition |
|---|---|---|---|
| HAS3 | F | 5′-CTTAAGGGTTGCTTGCTTGC-3′ | After polymerase activation at 94º C. |
| | R | 5′-GTTCGTGGGAGATGAAGGAA-3′ | for 5 minutes, polymerization |
| AQP3 | F | 5′-GTCACTCTGGGCATCCTCAT-3′ | reaction was performed in 40 cycles |
| | R | 5′-CTATTCCAGCACCCAAGAAGG-3′ | under conditions of 95° C. for 30 |
| β-actin | F | 5′-GGCCATCTCTTGCTCGAAGT-3′ | seconds, 55° C. for 30 seconds, |
| | R | 5′-GAGACCTTCAACACCCCAGC-3′ | and 72º C. for 30 seconds. |

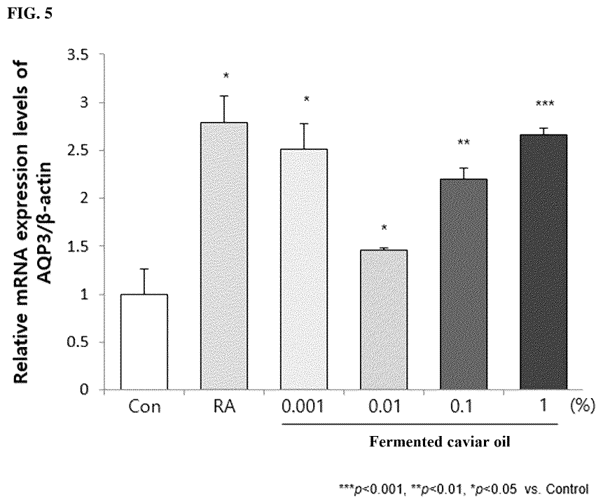

As a result, it was confirmed that an increase in HAS3 and AQP3 expression was promoted in cells treated with the fermented caviar oil, as shown in FIGS. 4 and 5, and through this, the skin moisturizing effect of the fermented caviar oil was confirmed.

### Experimental Example 4: Confirmation of Barrier Strengthening Effect

In order to confirm the barrier strengthening effect of the fermented caviar oil according to Example 1, it was evaluated by measuring the cohesion of the stratum corneum in keratinocytes and the gene expression of the main components Claudin1 and Filaggrin. Keratinocyte HaCaT was dispensed into a 6-well plate at an amount of 4×10⁵ cell/well and then cultured in an incubator at 37° C. and 5% CO₂ for 24 hours. Thereafter, the fermented caviar oil was added and further cultured for 24 hours. In this case, 1 μM of retinoic acid was used as a positive control. Thereafter, RNA was isolated from cells treated with each sample by using Trizol (RNA iso, DAKARA, Japan), RNA was quantified by using nanodrop, and then cDNA was synthesized in an amplifier using 2 μg of RNA, respectively (C1000 Thermal Cycler, Bio-Rad, USA).

Real-time polymerase chain reaction was performed on a real-time PCR machine using a mixture of Claudin1 and Filaggrin primers, target proteins, and SYBR Green (SYBR Green supermis, Applied Biosystems, USA), a cyanine dye, added to the synthesized cDNA, thereby finally evaluating the expression levels of the Claudin1 gene and the Filaggrin gene. The sequences and reaction conditions of the primer are shown in Table 5, and the gene expression levels were finally analyzed through correction for the β-actin gene.

TABLE 5

| Primer | | Sequence | Reaction condition |
|---|---|---|---|
| Claudin 1 | F | 5′-GCTCTAGAATTCCGAGCGAGTCATGGCCAAC | After polymerase |
| | | GC-3′ | activation at 94° C. for 5 |
| | R | 5′-GCTCTAGAATTCTCACACGTAGTCTTTCCCGC | minutes, polymerization |
| | | T-3′ | reaction was performed |
| Filaggrin | F | 5′-AGTGCACTCAGGGGGCTCACA-3′ | in 40 cycles under |
| | R | 5′-CCGGCTTGGCCGTAATGTGT-3′ | conditions of 95º C. for 30 |
| β-actin | F | 5′-GGCCATCTCTTGCTCGAAGT-3′ | seconds, 55º C. for 30 |
| | R | 5′-GAGACCTTCAACACCCCAGC-3′ | seconds, and 72° C. for 30 |
| | | | seconds. |

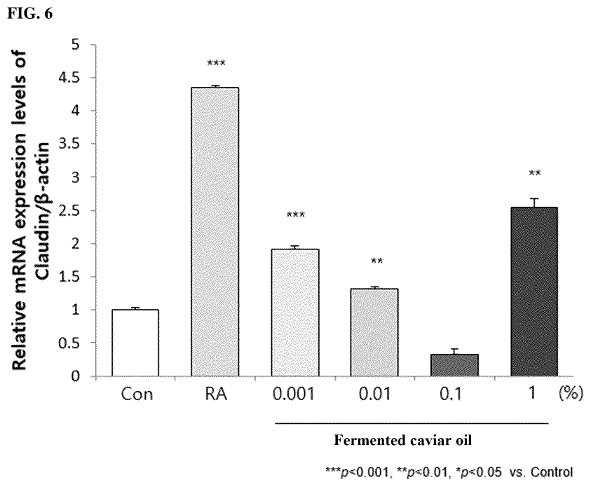

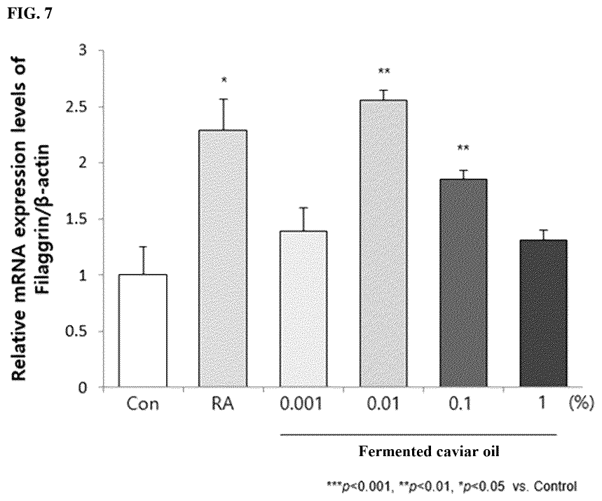

As a result, it was confirmed that an increase in Claudin1 and Filaggrin expression was promoted in cells treated with the fermented caviar oil, as shown in FIGS. 6 and 7, and through this, the skin barrier strengthening effect of the fermented caviar oil was confirmed.

### Experimental Example 5: Confirmation of Differentiation of Keratinocytes

The effect of the fermented caviar oil according to Example 1 on differentiation of keratinocytes was confirmed. The degree of differentiation was evaluated by measuring the gene expression of Involucrin and Loricrin, which are overexpressed when keratinocytes are differentiated.

Keratinocyte HaCaT was dispensed into a 6-well plate at an amount of 4×10⁵ cell/well and then cultured in an incubator at 37° C. and 5% CO₂ for 24 hours. Thereafter, the fermented caviar oil was added and further cultured for 24 hours. In this case, 1 μM of retinoic acid was used as a positive control. Thereafter, RNA was isolated from cells treated with each sample by using Trizol (RNA iso, DAKARA, Japan), RNA was quantified by using nanodrop, and then cDNA was synthesized in an amplifier using 2 μg of RNA, respectively (C1000 Thermal Cycler, Bio-Rad, USA).

Real-time polymerase chain reaction was performed on a real-time PCR machine using a mixture of Involucrin and Loricrin primers, target proteins, and SYBR Green (SYBR Green supermis, Applied Biosystems, USA), a cyanine dye, added to the synthesized cDNA, thereby finally evaluating the expression levels of the Involucrin gene and the Loricrin gene. The sequences and reaction conditions of the primer are shown in Table 6, and the gene 5 expression levels were finally analyzed through correction for the β-actin gene.

TABLE 6

| Primer | | Sequence | Reaction condition |
|---|---|---|---|
| Involucrin | F | 5′-TGAAACAGCCAACTCCACTG-3′ | After polymerase activation at 94° C. |
| | R | 5′-GGAGCTCCAACAGTTGCTCT-3′ | for 5 minutes, polymerization |
| Loricrin | F | 5′-AATAGATCCCCCAGGGTACCA-3′ | reaction was performed in 40 cycles |
| | R | 5′-CGGTGCCCCTGGAAAAC-3′ | under conditions of 95º C. for 30 |
| β-actin | F | 5′-GGCCATCTCTTGCTCGAAGT-3′ | seconds, 55° C. for 30 seconds, and |
| | R | 5′-GAGACCTTCAACACCCCAGC-3′ | 72º C. for 30 seconds. |

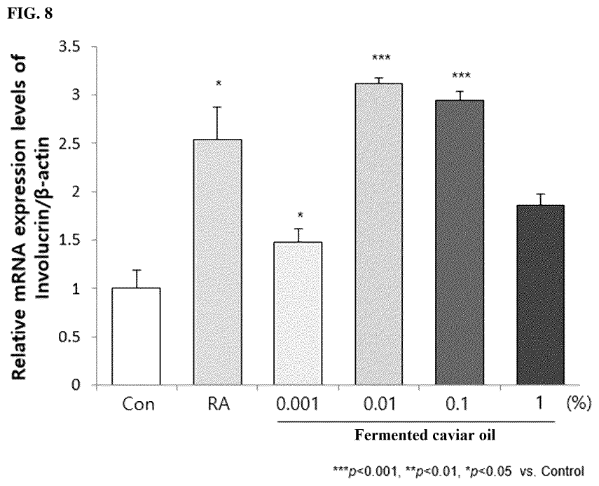

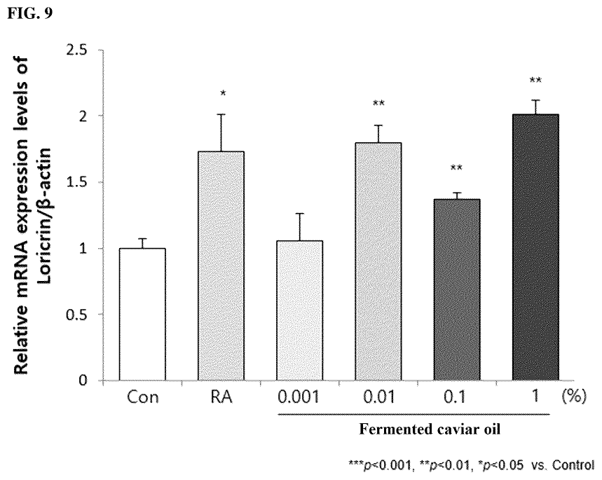

As a result, it was confirmed that an increase in Involucrin and Loricrin expression was promoted in cells treated with the fermented caviar oil, as shown in FIGS. 8 and 9, and through this, the effect of the fermented caviar oil on keratinocyte differentiation was confirmed.

### Experimental Example 6: Confirmation of Amino Group of Fermented Caviar Oil

For the fermented caviar oil of Example 1, it was confirmed using a Ninhydrin reaction test method whether fermentation lipid metabolites with amino groups were produced. Specifically, a sample of fermented caviar oil extracted with chloroform-methanol (2:1, v/v) was reacted with a 0.1% Nihydrin solution (solvent: ethanol). The mixing ratio was sample 2:0.1% Ninhydrin solution 1, and the reaction was performed under the condition of heating in a 90° C. water bath for 10 minutes. Thereafter, it was left to cool at room temperature for 10 minutes, and the absorbance was measured at 570 nm using a spectrophotometer (BioTek, USA).

TABLE 7

| | Comparative | | | |
|---|---|---|---|---|
| | Example 1 | Example 1 | Example 2 | Example 3 |
| O.D 570 nm | 0.070 | 0.515 | 0.778 | 0.850 |

As a result, the color development of ninhydrin was higher in Examples 1 to 3 than in Comparative Example 1. The caviar extract, which is a nitrogen source, improved the production of a lipid fermentation metabolite having an amino group, which is a substance involved in sphingo lipid metabolism and ceramide synthesis.

As described above, it was confirmed that the composition containing fermented caviar oil of the present invention has the effect of improving aging by suppressing MMP-1 expression in skin fibroblasts and promoting COL1A1 expression. In addition, by treating keratinocytes with the composition according to the present invention, a moisturizing effect was confirmed through increased expression of Hyaluronan Synthase 3 and Aquaporin 3, which are skin moisturizing factors, and an effect of improving the skin barrier was confirmed through accelerated expression of Claudin and Filaggrin. In addition, the excellent skin cell differentiation effect of the fermented caviar oil was confirmed through promoted expression of Involucrin and Loricrin.

The specific contents of the present invention have been described in detail above, and for those skilled in the art, these specific descriptions are merely preferred embodiments, and thus the scope of the present invention is not limited to the specific embodiments described above.

### [Microorganism Deposit Certificate]

1. A method for producing a cosmetic composition containing fermented caviar oil, the method including:
a seed culture step of producing a seed culture by culturing microorganisms with deposit number of KCCM 11843P (Korean Culture Center of Microorganisms (Overseas), 2016.06.08) as skin flora;
a preculture step of culturing the seed culture to produce a preculture; and
a main fermentation step of producing a fermented product by adding the preculture to a medium containing caviar extract.

2. The method for producing a cosmetic composition containing fermented caviar oil according to claim 1, wherein the medium of the seed culture step or the medium of the preculture step contains casein hydrolyzate, yeast extract, glucose, soluble starch, K₂HPO₄, MgSO₄, sodium pyruvate, and casein peptone.

3. The method for producing a cosmetic composition containing fermented caviar oil according to claim 1, wherein the medium of the seed culture step or the medium of the pre-culture step contains 0.1 to 5 g/L of casein hydrolysate, 0.01 to 10 g/L of yeast extract, 0.1 to 10 g/L of glucose, 0.1 to 5 g/L of soluble starch, 0.1 to 5 g/L of K₂HPO₄, 0.1 to 10 g/L of MgSO₄, 0.05 to 5 g/L of sodium pyruvate, and 0.1 to 5 g/L of casein peptone.

4. The method for producing a cosmetic composition containing fermented caviar oil according to claim 1, wherein the medium of the main fermentation step contains glycerol, yeast extract, casein peptone, K₂HPO₄, KH₂PO₄, and ammonium acetate.

5. The method for producing a cosmetic composition containing fermented caviar oil according to claim 1, wherein the medium of the main fermentation step contains 1 to 100 g/L of glycerol, 0.01 to 10 g/L of yeast extract, 0.1 to 10 g/L of casein peptone, 0.5 to 50 g/L of K₂HPO₄, 0.3 to 30 g/L of KH₂PO₄, 0.005 to 0.1 g/L of ammonium acetate, and 5 to 250 g/L of caviar extract.

6. The method for producing a cosmetic composition containing fermented caviar oil according to claim 1, wherein the medium of the main fermentation step further contains vegetable oil.

7. The method for producing a cosmetic composition containing fermented caviar oil according to claim 6, wherein the content of the vegetable oil is 5 to 500 g/L.

8. The method for producing a cosmetic composition containing fermented caviar oil according to claim 1, wherein the seed culturing step includes shaking culturing under aerobic conditions of 15 to 35° C.,
the preculture step includes adding 10 to 500 g/L of the seed culture and culturing under aerobic conditions of 15 to 35° C., 5 to 20 NL/min, and 10 to 100 rpm, and
the culture in the preculture step is terminated when the spectrophotometric absorbance of the 5% culture solution at a wavelength of 600 nm is 0.2 to 0.8.

9. The method for producing a cosmetic composition containing fermented caviar oil according to claim 1, wherein the main fermentation step includes adding 10 to 500 g/L of the preculture and fermenting it for 50 to 150 hours under aerobic conditions of 15 to 35° C., 100 to 500 NL/min, and 100 to 300 rpm.

10. The method for producing a cosmetic composition containing fermented caviar oil according to claim 1, wherein the composition is a composition for skin anti-aging, skin barrier strengthening, or skin moisturizing.

11. A cosmetic composition containing fermented caviar oil, produced by the method according to claim 1.


SEQUENCE LISTING (18 sequences)

SEQ ID NO: 1 - DNA, 20 bp, synthetic construct
  source 1..20: mol_type = other DNA
cgaattgccgacagagatga

SEQ ID NO: 2 - DNA, 22 bp, synthetic construct
  source 1..22: mol_type = other DNA
gtccctgaacagcccagtactt

SEQ ID NO: 3 - DNA, 20 bp, synthetic construct
  source 1..20: mol_type = other DNA
ggccatctcttgctcgaagt

SEQ ID NO: 4 - DNA, 20 bp, synthetic construct
  source 1..20: mol_type = other DNA
gagaccttcaacaccccagc

SEQ ID NO: 5 - DNA, 20 bp, synthetic construct
  source 1..20: mol_type = other DNA
ctcgaggtggacaccaccct

SEQ ID NO: 6 - DNA, 20 bp, synthetic construct
  source 1..20: mol_type = other DNA
cagctggatggccacatcgg

SEQ ID NO: 7 - DNA, 20 bp, synthetic construct
  source 1..20: mol_type = other DNA
cttaagggttgcttgcttgc

SEQ ID NO: 8 - DNA, 20 bp, synthetic construct
  source 1..20: mol_type = other DNA
gttcgtgggagatgaaggaa

SEQ ID NO: 9 - DNA, 20 bp, synthetic construct
  source 1..20: mol_type = other DNA
gtcactctgggcatcctcat

SEQ ID NO: 10 - DNA, 21 bp, synthetic construct
  source 1..21: mol_type = other DNA
ctattccagcacccaagaagg

SEQ ID NO: 11 - DNA, 33 bp, synthetic construct
  source 1..33: mol_type = other DNA
gctctagaattccgagcgagtcatggccaacgc

SEQ ID NO: 12 - DNA, 33 bp, synthetic construct
  source 1..33: mol_type = other DNA
gctctagaattctcacacgtagtctttcccgct

SEQ ID NO: 13 - DNA, 21 bp, synthetic construct
  source 1..21: mol_type = other DNA
agtgcactcagggggctcaca

SEQ ID NO: 14 - DNA, 20 bp, synthetic construct
  source 1..20: mol_type = other DNA
ccggcttggccgtaatgtgt

SEQ ID NO: 15 - DNA, 20 bp, synthetic construct
  source 1..20: mol_type = other DNA
tgaaacagccaactccactg

SEQ ID NO: 16 - DNA, 20 bp, synthetic construct
  source 1..20: mol_type = other DNA
ggagctccaacagttgctct

SEQ ID NO: 17 - DNA, 21 bp, synthetic construct
  source 1..21: mol_type = other DNA
aatagatcccccagggtacca

SEQ ID NO: 18 - DNA, 17 bp, synthetic construct
  source 1..17: mol_type = other DNA
cggtgcccctggaaaac